(19) United States
(12) Patent Application Publication
(10) Pub. No.: US 2003/0144632 A1
(43) Pub. Date: Jul. 31, 2003

(54) Injection device

(76) Inventors: Edgar Hommann, Grossaffoltern (CH); Hans-Christian Ruehrnschopf, Bern (CH); Ian Thompson, Burgdorf (CH); Markus Tschirren, Burgdorf (CH)

Correspondence Address: David E. Bruhn, DORSEY & WHITNEY LLP, Suite 1500, 50 South Sixth Street, Minneapolis, MN, 55402-1498, US

(21) Appl. No.: 10/354,864

(22) Filed: Jan. 30, 2003

(30) Foreign Application Priority Data
Jan. 30, 2002 (DE) 102 03 597.0

(51) Int. Cl. A61M 5/32

(52) U.S. Cl. 604/198

(57) ABSTRACT

The invention relates to an injection device including a tubular casing for a reservoir (ampoule) for a medicinal fluid, an injection needle, and a needle protecting sleeve which may be axially shifted on the casing between a protective position in which it surrounds the injection needle and a functional position in which it exposes the injection needle, wherein, in the protective position, the distal end of the needle protecting sleeve axially abuts at least one stopper and, in order to release the sleeve, the at least one stopper is radially shifted by applying a force to the casing.

### PRIORITY CLAIM

This utility patent application claims the priority of German Patent Application No. 102 03 597.0, filed on Jan. 30, 2002, which is incorporated in its entirety by reference.

### BACKGROUND

The invention relates to an injection device comprising a needle protecting sleeve which in a protective position surrounds an injection needle of the injection device and in a functional position exposes the injection needle. The application claims the priority of German patent application No. 102 03 597.0, filed on Jan. 30, 2002 with the German Patent and Trademark Office.

An injection device comprising a needle protecting device is known from U.S. Pat. No. 5,609,577 in which the injection needle is surrounded by a needle protecting sleeve. The needle protecting sleeve is supported on a casing of the injection device, axially shiftably. In the protective position, the needle protecting sleeve is secured by a block against the injection needle being unintentionally shifted or exposed. Once the block is released by turning the needle protecting sleeve relative to the casing, the needle protecting sleeve can be shifted into a rear, distal position in which the injection needle freely protrudes beyond the retracted needle protecting sleeve. When the needle protecting sleeve is retracted, a tension spring of the needle protective device is tensioned. When the needle protecting sleeve is relaxed after the injection, the needle protecting sleeve snaps back forward into its protective position under the tension stress of the tension spring. The blocking mesh or connection is automatically re-established in the protective position.

In this known needle protecting device, the tension spring is an essential element of the block. By using a spring to secure the needle protecting sleeve in the protective position, an elaborate design is required for the blocking mechanism. Furthermore, the proper functioning of the blocking mechanism is substantially dependent on the spring. Thus, the spring can break or jam. In the event of the spring malfunctioning, a secure block for the needle protecting sleeve in the protective position cannot be guaranteed.

Furthermore, a needle protecting device comprising a sleeve-shaped base body and a needle protecting sleeve follows from WO 01/64271. The needle protecting sleeve is supported on the base body, axially shiftably, and the base body can be either connected non-shiftably to a casing of the injection device or formed by a casing of the injection device. In the protective position, the needle protecting sleeve is blocked against being retracted relative to the base body and to the injection needle, by a blocking mesh or connection between a first blocking means associated with or attached to the needle protecting sleeve and a second blocking means associated with or connected to the base body. The first blocking means is formed by an elastically bending latch which abuts the second blocking member in the axial direction, said second blocking member forming a latch stopper. The blocking mesh of the two blocking means is released by bending the elastically ductile latch.

Admittedly, in this design, only two elements are required for the secure block, namely the elastically bending latch and the corresponding blocking stopper, while a third element, for example, in the form of a tension spring, is no longer required. However, the handling when releasing the block is somewhat awkward, since in one embodiment two cams must be axially shifted on opposing sides of the needle protecting sleeve.

Furthermore, an injection device follows from U.S. Pat. No. 5,282,793 in which an ampoule comprising an injection needle is supported in a casing, axially shiftably, and can be latched in a position in which the injection needle is situated completely within the casing, which in this state serves to protect the needle.

Furthermore, an injection device is known from U.S. Pat. No. 6,228,067 in which a protective cap for the needle can be manually removed.

U.S. Pat. No. 5,292,314 shows an injection device comprising a needle holder having an injection needle into which an ampoule for a medicinal fluid can be inserted, and comprising an axially shiftable needle protecting sleeve which is pre-tensioned by a helical spring into a protective position in which it surrounds the injection needle. The needle protecting sleeve is latched in the protective position, and the latch can be released by a rotational or longitudinal movement of the needle protecting sleeve, which makes it awkward to operate. In addition, two springs are provided in this needle protecting device, assisting in latching or releasing the needle protecting sleeve, respectively. Thus, this design is very elaborate.

### SUMMARY

The invention is based on the object of providing an injection device in which the disadvantages mentioned above do not occur. In particular, the intention is to propose an injection device which, despite an extremely simple and therefore fault-resistant design, can be handled easily.

The advantages achieved using the invention are based on a very simple design, since no additional elements are required on the side of the needle protecting sleeve, but rather the needle protecting sleeve is designed such that it serves as a stopper itself. For this purpose, a protrusion, an attachment, a projection or nose or a similar element can be associated with the sleeve, for example, be formed as one piece with the needle protecting sleeve, for abutting the stopper of the casing.

In one embodiment, the invention provides an injection device including a tubular casing for a reservoir (ampoule) for a medicinal fluid, an injection needle, and a needle protecting sleeve which may be axially moved on the casing between a protective position in which the injection needle is covered and a functional position in which the injection needle is exposed, wherein, in the protective position, the distal end of the needle protecting sleeve axially abuts at least one stopper associated with the casing and, in order to move the sleeve, the at least one stopper is radially shifted by applying a force to the casing.

In accordance with one particularly expedient embodiment, however, the free end face of the needle protecting sleeve itself can serve as an abutting surface, such that no modification of the needle protecting sleeve is required.

The counter piece, i.e., the stopper associated with the casing, can likewise be formed by at least one protrusion, attachment or projection or nose formed as one piece with the casing, such that no additional element is required here either. More than one stopper may be used.

In the protective position, the end face of the needle protecting sleeve abuts the stopper of the casing, such that the needle protecting sleeve is latched in this position, in which it surrounds the injection needle.

In order to release this latch, a force is exerted on the casing, perpendicular to the direction of movement of the needle protecting sleeve, such that the stopper of the casing moves radially and the needle protecting sleeve can therefore be moved backwards past the stopper, thus exposing the injection needle.

Once the medicinal fluid has been injected, the needle protecting sleeve can be pulled back forwards into the protective position by hand. In accordance with one preferred embodiment, however, a spring is used for this purpose, in particular a helical spring, which rests on the reservoir for the medicine fluid and is supported on the axial inner surface of the needle protecting sleeve. Against the force of this spring, the needle protecting sleeve has to be shifted out of the protective position and into the functional position in which it exposes the injection needle, such that the needle protecting sleeve automatically moves back into the protective position through the force of the spring and is latched there by the projection or nose on the casing locking back in behind the end face of the needle protecting sleeve.

Various technologies may be used for adjusting the casing to release the latch, for example, a shifting movement or also a sliding movement over a ramp.

In accordance with one preferred embodiment, however, a part of the gripping region of the casing is designed such that it is formed as one piece with the latching stopper and can be tilted about an axis running at right angles to the longitudinal axis of the injection device or the casing.

In accordance with one preferred embodiment, only narrow, strip-like parts of the gripping region of the casing are pivoted, while the rest of the gripping region is rigidly connected to the casing. The rigid part of the gripping region serves as a guide for the tilting parts situated top and bottom in the gripping region. The pivoting parts are preferably connected to the casing via a pin/hole joint.

Although it is in principle also possible to radially re-adjust the tilting parts of the gripping region manually, i.e., by exerting a corresponding force using ones finger, a spring is respectively provided between the stationary part of the casing and the pivoting parts of the gripping region in accordance with one preferred embodiment, said spring radially pre-tensioning the tilting part of the pivoting region such that the tilting part of the gripping region can return to its initial position as soon as the needle protecting sleeve has reached its protective position again, which automatically latches the needle protecting sleeve in this position.

A leaf spring, let into, for example, molded into, the tilting part of the casing at one end and resting on the outer surface of the rigid part of the casing at the other end, respectively serves as the pre-tensioning spring for the tilting parts of the casing. As an alternative to the above variant, the springs can also serve to fix the tilting parts.

The reservoir for the medicine fluid to be injected can be formed, for example, by a pre-filled ampoule provided with the piston for injecting the medicine fluid.

Said ampoule is formed as a disposable part, while the casing comprising the integrated needle protecting sleeve can be re-used. The ampoule is inserted into the casing from one end, the needle protecting sleeve being situated in the protective position and therefore surrounding the injection needle of the ampoule.

By releasing the latch between the casing and the needle protecting sleeve as explained, the needle protecting sleeve can be shifted into the functional position, wherein the injection needle is injected simultaneously or in a subsequent procedure. The medicinal fluid is then injected by operating the piston at the rear end of the ampoule.

### BRIEF DESCRIPTION OF THE DRAWINGS

The invention is explained in the following in more detail by way of examplary embodiments and by referring to the accompanying drawings.

FIG. 1 is a perspective view of the injection device with an inserted ampoule for a medicinal fluid;

FIG. 2 is a vertical section through the injection device with the needle protecting sleeve situated in the protective position;

FIG. 3 is a view corresponding to FIG. 2, but the needle protecting sleeve is situated in the functional position and the injection needle of the ampoule is thus exposed; and

FIG. 4 is a sectional view along line 4-4 from FIG. 2, with a view of the pivoting parts being fixed using a pin/hole joint.

### DETAILED DESCRIPTION

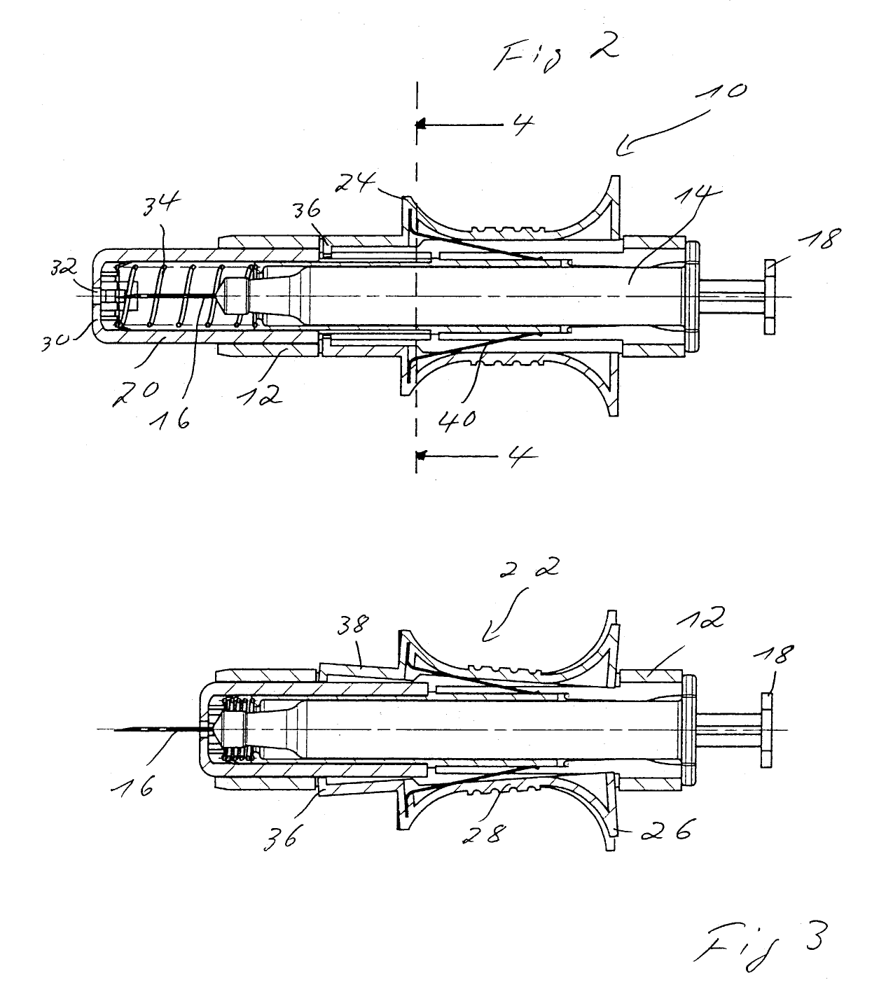

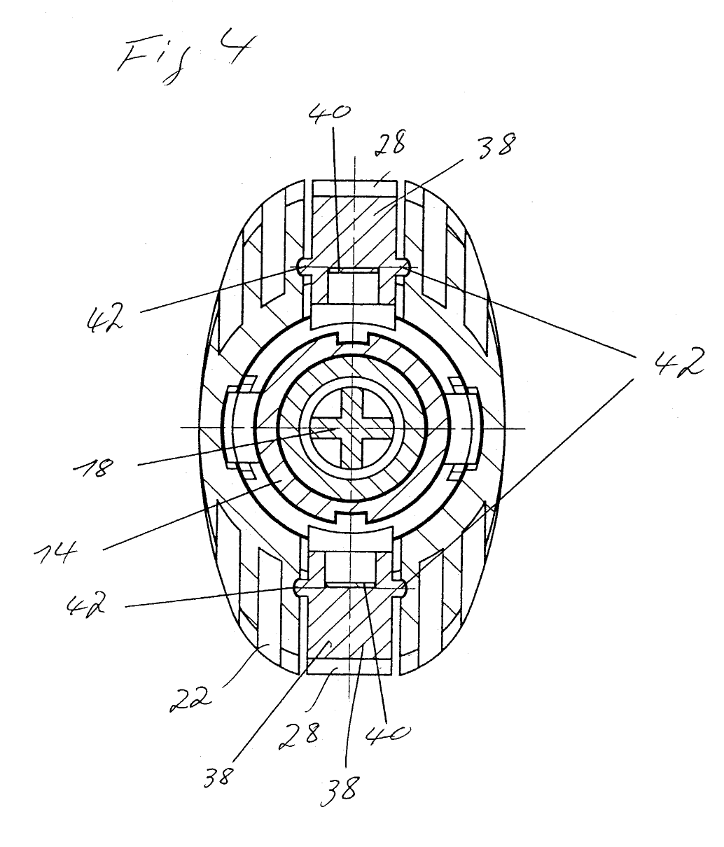

The injection device as may be seen from the figures, indicated as a whole by the reference numeral 10, substantially consists of three parts, namely a casing 12, an ampoule 14 having an injection needle 16 and a discharge piston 18, and a needle protecting sleeve 20 which in a protective position (see FIG. 2) surrounds the injection needle 16 and in the functional position (see FIG. 3) exposes it.

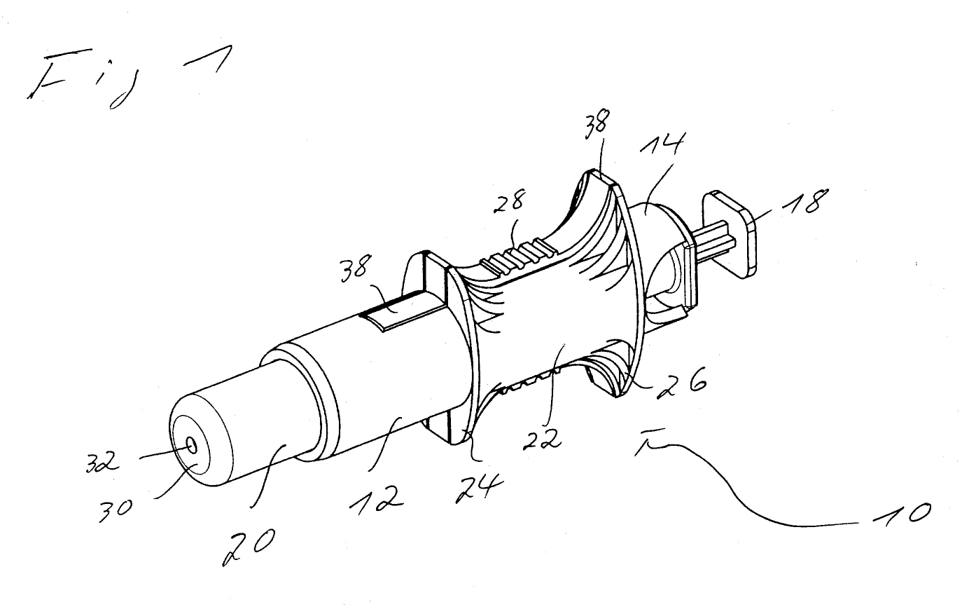

The ampoule 14 is formed as a disposable part and is inserted into the casing 12 from the right in accordance with the representation in FIG. 1, i.e., from the distal end of the casing.

The casing 12 substantially has the shape of a hollow cylinder and is provided with a gripping region 22 in its central part, said region being bordered to the front and to the back, in each case by a stay 26. Between the two stays 24, 26, the gripping region 22 has a rounded, roughly semi-elliptical shape top and bottom, wherein a ribbing 28 is situated at the lowest point of said rounded region.

In this way, the gripping region 22 can be gripped at the two ribbings 28 between the index and middle finger of a hand and the piston 18 simultaneously pushed in by the thumb of the same hand, such that it is possible to operate the injection device 10 with one hand.

The needle protecting sleeve 20 has the shape of a cylinder which is closed at one end, namely the left-hand end in accordance with the representation in the three figures, i.e., the proximal end, and is open at the opposite, distal end. The proximal end face 30 of the needle protecting sleeve is provided with an opening 32 through which the injection needle 16 (see FIG. 3) can emerge.

A helical pressure spring 34 is inserted into the needle protecting sleeve 20, said spring being fixed to the inner side of the end face 30 via its proximal end, the left-hand end in accordance with the representation in the figures, and abutting the proximal end face of the ampoule 14 at its distal end. The helical pressure spring 34 thus surrounds the injection needle 16 and the front, cylindrical part of the ampoule 14.

As may be seen in particular in FIG. 2, the needle protecting sleeve 20 protrudes into the proximal end of the casing 12 and via its distal end face abuts projections or noses 36 which protrude radially inwards and are situated at the proximal end of strip-like parts of the gripping region 22 which may be tilted relative to the casing 12. These tilting regions 38 also include the ribbing 28 already explained above.

The strip-like regions 38 are inserted into gaps of a corresponding width in the two stays 24, 26 and top and bottom in the casing 12, as shown in FIGS. 2 and 3, and their inner sides are fixed to the casing 12, in each case via a leaf spring 40 which is attached to the casing 12 via its distal end and to the proximal stay 24 via its proximal end, and in particular is let into the stay 24.

As an alternative to fixing the strip-like, ductile parts 38 of the gripping region 22 as explained above, the ductile, strip-like parts 38 can also be connected, in the region of their tilting axis, to the rigid, stationary casing 12 via a ductile coupling situated roughly in the region of the tilting axes.

FIG. 4 shows another alternative way of fixing, namely via a pin/hole joint. As may be seen from the vertical section through the gripping region along line 4-4 from FIG. 2, small pin-like protrusions 42 are provided on the lateral edges of the strip-like parts 38 of the gripping region 22 near the proximal stay 24, said protrusions being formed as one piece with the strip-like, ductile parts 38, for example, when made from a plastic, being simultaneously molded with said parts. The pins 42 are clipped into the gripping region 22 in recesses on the inner edges of the strip-like recess, which in turn accommodates the strip-like, ductile parts 38 top and bottom.

In this way, the strip-like, ductile parts 38 top and bottom can thus be pivoted about the two pin/hole joints formed by the protrusions 42 and their recesses.

The shape of the leaf springs 40 and their attachment to the casing 12 on the one hand and to the stay 24 of the gripping region 22 on the other are adapted to each other such that the leaf springs 40 pre-tension the strip-like parts 38 into the position which can be seen in FIGS. 1 and 2, in which the proximal ends of the strip-like regions 38 are flush on their outer surfaces with the outer surface of the casing 12, and the two projections or noses 36 are situated in the axial movement path of the needle protecting sleeve 20, i.e., the distal end face of the needle protecting sleeve 20 abuts the projections or noses 36.

By exerting a force on the tilting strip-like parts 38 of the gripping region 22 using two fingers of one hand, the strip-like parts 38 are tilted against the force of the leaf spring 40 about their joint axes situated roughly below or above the proximal stay 24 of the gripping region 22, clockwise (top) or anti-clockwise (bottom) in accordance with the representation in FIGS. 2 and 3.

The tilting movement of the strip-like regions 38 pivots the projections or noses 36 radially outwards, out of the position shown in FIG. 2 in which the distal end of the needle protecting sleeve 20 abuts the projections or noses 36 and the needle protecting sleeve 20 is therefore situated in the protective position, and into the position shown in FIG. 3 in which the needle protecting sleeve 20 can be shifted into the position shown in FIG. 3 once the latch has been released by the projections or noses 36.

This can be achieved, against the force of the pressure spring 34, either by manually shifting the needle protecting sleeve 20 or by placing the front surface of the needle protecting sleeve 20 onto a point on the body into which the medicine fluid is to be injected and pressing the needle protecting sleeve into the casing 12. The injection needle is simultaneously or subsequently injected into the point on the body.

If the medicinal fluid is then discharged from the ampoule 14 via the needle 16 by pressing in the piston 18, and so injected into the body of the patient, the manual pressure on the resting point of the needle sleeve 20 can then be released, returning the needle sleeve 20 from the functional position (see FIG. 3) to the protective position (see FIG. 2) through the force of the pressure spring 34.

The needle protecting sleeve 20 moves through under the two projections or noses 36 until its distal end has moved past the projections or noses 36.

Due to the force of the leaf springs 40, the projections or noses 36 are then pivoted by the tilting movement of the strip-like parts 38 radially inwardly, clockwise (top) or anti-clockwise (bottom), the projections or noses 36 thereby meshing or connecting with or lodging against the distal end of the needle protecting sleeve 20 and so latching or releasably locking it in the protective position (see FIG. 2).

The ampoule 14, together with the injection needle 16 and the piston 18, can then be removed from the casing 12 and disposed of.

The ampoule 14 has the conventional function wherein the ampoule 14 is expediently fixed in the casing 12 by a latch which is easily overcome and which operates via a positive lock and/or a frictional lock, in order that the ampoule 14 cannot fall out when the casing 12 is positioned obliquely. Any suitable technology or structure may be used for this purpose.

In the foregoing description, embodiments of the invention, including preferred embodiments, have been presented for the purpose of illustration and description. They are not intended to be exhaustive or to limit the invention to the precise form disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. An injection device, comprising:
a) a tubular casing for a reservoir for a medicinal fluid;
b) an injection needle;
c) a needle protecting sleeve which may be axially shifted on said casing between a protective position in which it surrounds said injection needle and a functional position in which the injection needle is exposed;
d) a means for latching said needle protecting sleeve in said protective position; and
e) a means for releasing said latch, such that the needle protecting sleeve may be shifted into said functional position; wherein:
f) in the protective position, the needle protecting sleeve axially abuts at least one stopper associated with the casing and is thus latched; and
g) in order to release the latch, the at least one stopper is shifted and released from abutting the needle protecting sleeve by applying a force to the casing perpendicular to the direction of movement of the needle protecting sleeve.

2. The injection device as set forth in claim 1, wherein the casing comprises a gripping region and wherein at least one part of the gripping region may be pivoted to release the latch.

3. The injection device as set forth in claim 2, wherein said gripping region comprises at least one pivoting, strip-like part having a projection serving as a stopper for a distally facing edge of the needle protecting sleeve.

4. The injection device as set forth claim 3, wherein said projection protrudes radially inwardly.

5. The injection device as set forth in claim 3, wherein at least one pivoting, strip-like part is pre-tensioned by a spring for a tilting movement into the position in which said projection abuts the distal end of the needle protecting sleeve.

6. The injection device as set forth in claim 5, wherein said spring is a leaf spring.

7. The injection device as set forth in claim 5, wherein the pivoting, strip-like part of the gripping region is fixed to the casing by means of said spring.

8. The injection device as set forth in claim 5, wherein the at least one pivoting, strip-like part of the gripping region is connected to the casing by a flexible coupling situated roughly in the region of the tilting axis.

9. The injection device as set forth in claim 3, wherein the pivoting part is supported via a complementary connector in the gripping region.

10. The injection device as set forth in claim 9, wherein complementary connector arrangement comprises a hole/pin arrangement.

11. The injection device as set forth in claim 1, further comprising a helical pressure spring between said reservoir and a proximal end of the needle protecting sleeve.

12. The injection device as set forth in claim 2, wherein the gripping region comprises two opposed, pivoting parts which can be adjusted.

13. An injection device comprising a tubular casing, an injection needle coupled to the casing, and a needle protecting sleeve which may be axially moved relative to the casing between a protective position in which the injection needle is covered and a functional position in which the injection needle is exposed, wherein, in the protective position, a distal end of the needle protecting sleeve axially abuts at least one stopper associated with the casing and, in order to move the sleeve to the functional position, the at least one stopper is radially shifted by applying a force to the casing.
